(19) United States
(12) Patent Application Publication
(10) Pub. No.: US 2004/0018984 A1
(43) Pub. Date: Jan. 29, 2004

(54) Methods for preventing adhesion formation using protease inhibitors

(76) Inventor: Mizuo Miyazaki, Takatsuki (JP)

Correspondence Address: DAVID R PRESTON & ASSOCIATES, 12625 HIGH BLUFF DRIVE, SUITE 205, SAN DIEGO, CA, 92130, US

(21) Appl. No.: 10/602,035

(22) Filed: Jun. 23, 2003

Related U.S. Application Data
(60) Provisional application No. 60/396,493, filed on Jul. 17, 2002.

(51) Int. Cl. A61K 38/06

(52) U.S. Cl. 514/18

(57) ABSTRACT

The present invention generally provides methods for the prevention or reduction of adhesion formation/reformation using protease inhibitors. More specifically, this invention provides methods for preventing or inhibiting postoperative adhesion formation/reformation in mammals following surgical or accidental injury or inflammation to the organs of the peritoneal or pleural cavity or other body spaces, using serine protease inhibitors, such as, for example, using chymase inhibitors (e.g., α-aminoalkylphosphonate derivatives) and the like.

### RELATED APPLICATIONS

The present application claims benefit of priority to provisional patent application serial No. 60/396,493, entitled “Methods for Preventing Adhesion Formation Using Protease Inhibitors” filed Jul. 17, 2002 which is incorporated by reference herein in its entirety.

### TECHNICAL FIELD OF THE INVENTION

The present invention relates generally to methods for the prevention of adhesion formation using protease inhibitors. More specifically, this invention relates to the prevention of postoperative adhesion formation using serine protease inhibitors, particularly chymase inhibitors.

### BACKGROUND OF THE INVENTION

Postoperative adhesion formation is an important complication following a variety of surgical procedures, including, for example, cardiac, thoracic, gynecologic, ophthalmic and general abdominal surgery. Pericardial adhesions pose a significant problem and increase morbidity and mortality of reoperative cardiac surgical procedures (Krause et al., J. Invest. Surg. 14:93-97, 2001). Postoperative peritoneal adhesions are a major source of morbidity following abdominal and gynecologic surgery, with the most serious complications being intestinal obstruction, infertility and pain (Liakakos et al., Dig. Surg. 18:260-73, 2001). While a number of factors have been implicated in adhesion formation including trauma, desiccation, ischemia, peritonitis of infectious origin and endometriosis (Holtz, Fertil. Steril. 41:497-507, 1984; Sharpe-Timms et al., Fertil. Steril. 69:916-23, 1998), the pathophysiological nature of adhesion formation is not completely understood.

Postsurgical adhesions generally result from the normal wound healing response and develop in the first five to seven days after injury. Adhesion formation and adhesion-free re-epithelialization are alternative pathways, both of which begin with coagulation, which initiates a cascade of events resulting in the buildup of fibrin gel matrix. If fibrin deposition is in excess or not removed, the fibrin gel matrix serves as the progenitor to adhesions by forming a band or bridge when two tissue surfaces coated with the fibrin matrix are apposed. The band or bridge becomes the basis for the organization of an adhesion by cellular elements, including fibroblasts. Protective fibrinolytic enzyme systems of the peritoneum, such as the plasmin system, can remove the fibrin gel matrix. However, surgery dramatically diminishes fibrinolytic activity. Therefore, pivotal events in determining whether the pathway taken is adhesion formation or re-epithelialization can include the apposition of two damaged tissue surfaces and the extent of fibrinolysis.

A variety of approaches toward the prevention and treatment of adhesion formation have been evaluated by others, with limited success or undesirable side effects (dizerega and Rogers, The Pertioneum, Springer-Verlag, N.Y. 307-369, 1992). For example, in an attempt to prevent fibrin deposition, peritoneal lavages to wash away fibrinous exudates, surgical techniques to minimize tissue ischemia, and utilization of barriers to limit apposition of healing serosal surfaces have been tested (DeChemey et al., Surg. Clin. North Am. 77:671-688, 1997; Liakakos et al., Dig. Surg. 18:260-73, 2001). Alternatively, anti-inflammatory drugs such as corticosteroids and nonsteroidals have been explored (Baysal, Clin. Exp. Obstet. Gynecol. 28:126-127, 2001). Finally, strategies to remove fibrin deposits after scaffold formation using conventional proteolytic enzymes and fibrinolytics have been considered (Hioki et al., Int. Surg. 83:11-14, 1998). Clearly, no single therapeutic approach has proven universally effective in preventing formation of postoperative adhesions.

Mast cells play an important role in the induction of allergic inflammatory responses. They express cell surface receptors for the Fc portions of IgE and for certain classes of IgG, enabling them to bind antibody to their surfaces. Interaction of antigen with the bound antibody results in the release from mast cells of a series of potent mediators such as histamine, serotonin, cytokines and a variety of enzymes that play critical roles in initiating allergic and anaphylactic-type responses. In addition, mast cells also have been suggested to be involved with peritoneal adhesion formation (Liebman et al., .M., Am. J. Surg. 165;127-130, 1993). Mast-cell stabilizers, which inhibit the activation and accumulation of mast cells, are effective in attenuating adhesion formation in rat models (Adachi et al., Surg. Today 29:51-54, 1999). We also demonstrated that adhesion formation in mast-cell-deficient mice was significantly less severe than that in normal control mice (Yao et al., J. Surg. Res. 92:40-44, 2000). These reports suggest that mast cells are closely related to adhesion formation, but the exact nature of such a role or which, if any, of the many factors released by mast cells might mediate this role has not been defined.

Chymase is a chymotrypsin-like serine protease contained in the secretory granules of mast cells. Chymase activates stem cell factor, a typical cytokine that has the ability to induce the accumulation of mast cells. In addition, chymase activity was significantly increased in healing sites after cecal scraping in mice (Yao et al., J. Surg. Res. 92:40-44, 2000). Thus, accumulation of chymase positive mast cells may play a role in the development of adhesion formation. However, any pathophysiological role of chymase in the development of adhesion formation remains unclear. To elucidate a possible relation between chymase and adhesion formation, we have now investigated the effect of using protease inhibitors, which have specific activity against chymase, as a means to prevent or inhibit adhesion formation

### SUMMARY OF THE INVENTION

There remains a significant need for methods to effectively prevent or reduce the adhesion formation that is a relatively common consequence following a variety of surgical procedures, including, for example, cardiac, thoracic, gynecologic, ophthalmic and abdominal surgeries. The present invention generally provides methods for the prevention or reduction of adhesion formation/reformation using protease inhibitors. More specifically, this invention provides methods for preventing or inhibiting postoperative adhesion formation/reformation in mammals following surgical or accidental injury or inflammation to the organs of the peritoneal or pleural cavity or other body spaces, using serine protease inhibitors, such as, for example, using chymase inhibitors (e.g., α-aminoalkylphosphonate derivatives) and the like.

In one aspect, methods are provided for preventing or reducing adhesion formation between tissue surfaces in a vertebrate subject, comprising administering to the subject an effective amount of at least one protease inhibitor to a site on a tissue surface for a period of time sufficient to permit tissue repair. In one embodiment, the method of preventing or reducing adhesion formation utilizes at least one protease inhibitor that is an inhibitor of a serine protease. In a preferred embodiment, the method utilizes at least one protease inhibitor that is an inhibitor of chymotrypsin-like serine proteases. Preferably, the vertebrate subject is a human.

In another aspect of the present invention, methods are provided for preventing or reducing adhesion formation between tissue surfaces in a vertebrate subject, comprising administering to the subject an effective amount of at least one chymase inhibitor to a site on a tissue surface for a period of time sufficient to permit tissue repair. In preferred embodiments, the method of preventing or reducing adhesion formation utilizes a chymase that is a peptidyl derivative of aryl diesters of α-aminoalkylphosphonic acids, such as for example Suc-Val-Pro-Phe^{P}(OPh)₂. In particularly preferred embodiments, the method utilizes an enantiomerically enriched preparation of a peptidyl derivative of aryl diesters of α-aminoalkylphosphonic acids. For example, in one enantiomerically enriched preparation, Suc-Val-Pro-Phe^{P}(OPh)₂ is utilized, wherein the most active enantiomer, Suc-Val-Pro-Phe^{P}(OPh)₂-A, comprises greater than 50%, 80% or even 95% by weight of the total Suc-Val-Pro-Phe^{P}(OPh)₂ in said enantiomerically enriched preparation. Preferably, the vertebrate subject is a human.

In another aspect of the present invention, methods are provided for preventing or reducing adhesion formation between tissue surfaces in a vertebrate subject, comprising administering to the subject an effective amount of at least one protease inhibitor to a site on a tissue surface for a period of time sufficient to permit tissue repair and wherein said protease inhibitor is administered to said subject before, during or after a surgical procedure. In preferred embodiments, the surgical procedure is an abdominal, gynecologic, cardiac, ophthalmic or thoracic surgical procedure.

In another aspect of the present invention, methods are provided for preventing or reducing postoperative adhesion formation in the peritoneum of a warm-blooded mammal, comprising administering to said mammal an effective amount of at least one serine protease inhibitor to a site on an organ surface for a period of time sufficient to permit tissue repair. In one preferred embodiment, the method of preventing or reducing postoperative adhesion formation utilizes at least one serine protease inhibitor that is an inhibitor of chymotrypsin-like serine proteases. Most preferably, said warm-blood mammal is a human.

In another aspect of the present invention, methods are provided for preventing or reducing postoperative adhesion formation in the peritoneum of a warm-blooded mammal, comprising administering to said mammal an effective amount of at least one chymase inhibitor to a site on an organ surface for a period of time sufficient to permit tissue repair. In preferred embodiments, the method of preventing or reducing adhesion formation utilizes a chymase inhibitor that is a peptidyl derivative of aryl diesters of α-aminoalkylphosphonic acids, for example Suc-Val-Pro-Phe^{P}(OPh)₂. In particularly preferred embodiments, the method utilizes an enantiomerically enriched preparation of Suc-Val-Pro-Phe^{P}(OPh)₂, wherein Suc-Val-Pro-Phe^{P}(OPh)₂-A comprises greater than 50%, 80% or even 95% by weight of the total Suc-Val-Pro-Phe^{P}(OPh)₂ in said enantiomerically enriched preparation. Preferably, said warm-blood mammal is a human.

In yet another aspect of the present invention, the above methods of preventing or reducing adhesion formation are provided, wherein said protease inhibitor is administered in conjunction with a delivery vehicle which maintains an effective local concentration of said protease inhibitor at said site, and wherein said delivery vehicle comprises microcapsules or microspheres. In one preferred embodiment, said microcapsules or microspheres comprise a biodegradable polymer selected from the group consisting of poly(α-hydroxy acids), polyhydroxybutyric acids, polycaprolactones, polyorthoesters, polyanhydrides, PACA, polycyanoacrylates, poly(D,L-lactide-co-glycolide) and mixtures thereof.

In yet another aspect of the present invention, the above methods of preventing or reducing adhesion formation are provided, wherein said protease inhibitor is administered in conjunction with a delivery vehicle which maintains an effective local concentration of said protease inhibitor at said site, and wherein said delivery vehicle comprises a film. In one preferred embodiment, said film comprises a biodegradable polymer selected from the group consisting of poly(α-hydroxy acids), polyhydroxybutyric acids, polycaprolactones, polyorthoesters, polyanhydrides, PACA, polycyanoacrylates, poly(D,L-lactide-co-glycolide) and mixtures thereof.

In yet another aspect of the present invention, the above methods of preventing or reducing adhesion formation are provided, wherein said protease inhibitor is administered in conjunction with a delivery vehicle which maintains an effective local concentration of said protease inhibitor at said site, and wherein said delivery vehicle comprises liposomes.

In yet another aspect of the present invention, the above methods of preventing or reducing adhesion formation are provided, wherein said protease inhibitor is administered in conjunction with a delivery vehicle which maintains an effective local concentration of said protease inhibitor at said site, and wherein said delivery vehicle comprises a high-molecular weight carrier selected from the group consisting of hyaluronic acid, hydrogels, carboxymethlcellulose, dextrans, cyclodextrans, and mixtures thereof.

In yet another aspect of the present invention, pharmaceutical compositions for the prevention of adhesion formation are provided, comprising any of the above protease inhibitors and a pharmaceutically acceptable diluent or excipient. In one embodiment, the pharmaceutical composition further comprises a delivery vehicle which maintains an effective local concentration of said protease inhibitor at a site on a tissue surface for a period of time sufficient to permit tissue repair.

These and other aspects and embodiments of the invention will become evident upon reference to the following detailed description and attached figures. In addition, various references set forth herein that describe in more detail certain procedures or compositions, and are therefore incorporated by reference in their entirety as if each were individually noted for incorporation.

### BRIEF DESCRIPTION OF FIGURES

FIG. 1 is a chromatograph showing enantiomer purification

FIG. 2 is a chromatograph showing enantiomer purification

FIG. 3 is histological samples showing an adhesion lesion between the oviduct and the small intestine stained with hematoxylin-eosin (A) and toluidine blue (B), and immunostained with an antibody to hamster chymase (C).

FIG. 4 is a graph showing significantly increased chymase activity in uterus extracts from placebo-treated hamsters as compared to control hamsters, and a corresponding decrease in chymase activity in uterus extracts from hamsters treated with a serine protease inhibitor as compared to the placebo-treated animals.

FIG. 5 is a table comparing scores of adhesion formation from placebo-treated hamsters versus hamsters treated with a serine protease inhibitor

FIG. 6 is a graph showing significantly reduced adhesion formation in hamsters treated with a serine protease inhibitor as compared to placebo-treated hamsters.

### DETAILED DESCRIPTION OF THE INVENTION

A. Definition of Terms

The following terms are used throughout the specification. Unless otherwise indicated, these terms are defined as follows:

The terms “effective amount” or “pharmaceutically effective amount” of a given composition, as provided herein, refer to a sufficient amount of the composition to provide a desired response, such as prevention or reduction in adhesion formation as outlined below, with little or no toxicity. As will be apparent, the exact amount required can vary from subject to subject, depending on the species, age, weight and general condition of the subject, and the particular protease of interest, mode of administration, and the like. An appropriate “effective” amount in any individual case may be determined readily by one of ordinary skill in the art using the teachings provided herein and routine methods.

By “vertebrate subject” is meant any member of the subphylum cordata, including, without limitation, mammals such as cattle, sheep, pigs, goats, horses, and humans; domestic animals such as dogs and cats; and birds.

By “pharmaceutically acceptable” or “pharmacologically acceptable” is meant any material, compound, composition or dosage form which, within the scope of sound medical judgment, is suitable for use in contact with the tissues of human beings or animals without excessive toxicity, irritation, allergic response, or other problem or complication, commensurate with a reasonable benefit/risk ratio.

As used herein, the terms “peritoneal cavity” or “peritoneum” and “abdominal cavity” or “abdomen” are used as synonyms, as are the terms “pleural” and “thoracic” cavity. The term “body spaces” is meant cavities or areas of the body where there is an environment for allowing the composition of the present invention to be in contact with body tissues. “Body spaces” may be further defined as peritoneal, abdominal, and thoracic cavities as well as joints and areas of the body containing synovial fluid.

As used herein, “treatment” (including variations thereof, for example, “treat” or “treated”) refers to administering one or more compositions with protease inhibitory activity in an effective amount to achieve reduction or elimination of adhesion formation. Treatment generally refers to a prophylactic or preventative method of achieving the desired outcome and may, in certain embodiments, be effected prior to, during or after a surgical event.

B. Protease Inhibitors

One preferred group of proteases, as targets of the present invention, are the serine proteases. Serine proteases are the subclass of endopeptidases that use serine as the nucleophile in peptide bond cleavage (Barrett, A. J., In: Proteinase Inhibitors, Ed. Barrett, A. J. et al, Elsevier, Amsterdam, pages 3-22, 1986). Serine proteases are well known in the art and two superfamilies of serine proteases, i.e., the chymotrypsin superfamily and the Streptomyces subtilisin superfamily, have been observed. Examples of serine proteases of the chymotrypsin superfamily include tissue-type plasminogen activator (hereinafter “t-PA”), trypsin, trypsin-like protease, chymotrypsin, plasmin, elastase, urokinase (or urinary-type plasminogen activator (hereinafter “u-PA”)), acrosin, activated protein C, Cl esterase, cathepsin G, chymase and proteases of the blood coagulation cascade including kallikrein, thrombin, and Factors VIIa, IXa, Xa, XIa and XIIa (Barrett, A. J., In: Proteinase Inhibitors, Ed. Barrett, A. J. et al, Elsevier, Amsterdam, pages 3-22 (1986); Strassburger, W. et al, FEBS Lett., 157:219-223 (1983)). The catalytic domains of all of the serine proteases of the chymotrypsin superfamily have both sequence homology and structural homology. The sequence homology includes the total conservation of: (i) the characteristic active site residues (e.g., Ser195, His57; and Asp102 in the case of trypsin); (ii) the oxyanion hole (e.g., Gly193, Asp194 in the case of trypsin); and (iii) the cysteine residues that form disulfide bridges in the structure (Hartley, B. S., Symp. Soc. Gen. Microbiol., 24:152-182 (1974)). The structural homology includes: (i) the common fold that consists of two Greek key structures (Richardson, J., Adv. Prot. Chem., 34:167-339 (1981)); (ii) a common disposition of catalytic residues; and (iii) detailed preservation of the structure within the core of the molecule (Stroud, R. M., Sci. AM., 231:24-88 (1974)).

Serine protease inhibitors are well known in the art and are divided into the following families: (i) the bovine pancreatic trypsin inhibitor (Kunitz) family, also known as basic protease inhibitor (Ketcham, L. K. et al, In: Atlas of Protein Sequence and Structure, Ed. Dayhoff, M. O., pages 131-143 (1978) (hereinafter “BPTI”), (ii) the Kazal family, (iii) the Streptomyces subtilisin inhibitor family (hereinafter “SSI”), (iv) the serpin family, (v) the soybean trypsin inhibitor (Kunitz) family, (vi) the potato inhibitor family, and (vii) the Bowman-Birk family (Laskowski, M. et al, Ann. Rev. Biochem., 49:593-626 (1980); Read, R. J. et al, In: Proteinase Inhibitors, Ed. Barrett, A. J. et al, Elsevier, Amsterdam, pages 301-336 (1986); and Laskowski, M. et al, Cold Spring Harbor Symp. Quant. Biol., LII:545-553 (1987)). Crystallographic data are available for a number of intact inhibitors including members of the BPTI, Kazal, SSI, soybean trypsin and potato inhibitor families, and for a cleaved form of the serpin alpha-1-antitrypsin (Read, R. J. et al, In: Proteinase Inhibitors, Ed. Barrett, A. J. et al, Elsevier, Amsterdam, pages 301-336 (1986)). Many of the serine protease inhibitors have a broad specificity and are able to inhibit both the chymotrypsin superfamily of proteases, including the blood coagulation serine proteases, and the Streptomyces subtilisin superfamily of serine proteases (Laskowski et al, Ann. Rev. Biochem., 49:593-626, 1980). The specificity of each inhibitor is thought to be determined primarily by the identity of the amino acid that is immediately amino-terminal to the site of potential cleavage of the inhibitor by the serine protease. This amino acid, known as the P site residue, is thought to form an acyl bond with the serine in the active site of the serine protease (Laskowski, M. et al, Ann. Rev. Biochem., 49:593-626 (1980)).

More preferred serine protease inhibitors for use in the present invention belong to the serpin family and the Bowman-Birk family. The serpin family serine protease inhibitors include the plasminogen activator inhibitors PAI-1, PAI-2 and PAI-3, Cl esterase inhibitor, alpha-2-antiplasmin, contrapsin, alpha-1-antitrypsin, antithrombin III, protease nexin I, alpha-1-antichymotrypsin, protein C inhibitor, heparin cofactor II and growth hormone regulated protein (Carrell et al, Cold Spring Harbor Symp. Quant. Biol., 52:527-535 (1987); Sommer et al, Biochem., 26:6407-6410 (1987); Suzuki et al, J. Biol. Chem., 262:611-616 (1987); and Stump et al, J. Biol Chem., 261:12759-12766 (1986)). The inhibition of serine proteases by serpins has been reviewed in Travis et al, Ann. Rev. Biochem., 52:655-709 (1983); Carrell, R. W. et al, Trends Biochem. Sci., 10:20-24 (1985); Sprengers, E. D. et al, Blood, 69:381-387 (1987); and Proteinase Inhibitors, Ed. Barrett, A. J. et al, Elsevier, Amsterdam (1986).

Preferred serine protease inhibitors for use in the present invention include peptidyl derivatives of aryl diesters of α-aminoalkylphosphonic acids. These α-aminoalkylphosphonate derivatives have be found to be excellent inhibitors of several serine proteases including bovine thrombin, human factor XIIa, human factor Xa, human plasma kallikrein, bovine trypsin, rat skin tryptase, human leukocyte elastase, porcine pancreatic elastase, bovine chymotrypsin, human leukocyte cathepsin G and rat mast cell protease II (U.S. Pat. Nos. 5,543,396; 5,686,419; 5,952,307; Oleksyszyn and Powers, Biochem., 30:485-493, 1991). These derivatives are remarkably stable under a variety of conditions, including in human plasma. For example, Suc-Val-Pro-Phe^{P}(OPh)₂, a particularly preferred inhibitor for use in the present invention, has a half-life of approximately 20 hr in human plasma. This stability is important because peritoneal exudates would be expected to neutralize most protease inhibitors of lesser stability, rendering them useless. The diesters of α-aminoalkylphosphonic acids are analogues of natural a-amino acids and are designated by the generally accepted three letter abbreviations for the amino acid followed by the superscript P. For example diphenyl α-(N-benzyloxycarbonylamino)ethylphosphonate which is related to alanine is abbreviated as Cbz-Ala^{P}(OPh)₂.

Peptidyl derivatives of aryl diesters of α-aminoalkylphosphonic acids inhibit serine proteases by reaction with the active site serine to form “phosphonylated” enzymes, which due to similarity of phosphorus atom to the tetrahedral intermediate formed during peptide hydrolysis, show remarkable stability. The enzyme catalytic apparatus is required to activate the phosphorus atom for nucleophilic substitution and reaction with enzyme. The activation is mainly due to precise interaction with the S1 pocket of various serine proteases. The phosphonate is believed to first bind to the enzyme and then react to form a covalent bond with the active site serine residue. Slow aging can take place with loss of the phenoxy group.

Peptides with a C-terminal phosphonate residue which is an analog of valine, such as Val^{P}(OPh)₂, are relatively potent and relatively specific irreversible inhibitors of elastase and elastase-like enzymes. The peptides with C-terminal phosphonate residues related to phenylalanine, other aromatic amino acids or amino acids with long aliphatic side chains are relatively potent and relatively specific inhibitors of chymotrypsin and chymotrypsin-like enzymes. The peptides with C-terminal phosphonate residues related to ornithine, lysine, arginine or containing a C-terminal diphenyl ester of α-amino-α-(4-amidinophenyl)methanephosphonate [(4-AmPhGly)^{P}(OPh)₂] or α-amino-α-(4-amidinophenylmethyl)methanephosphonate [(4-AmPhe)^{P}(OPh)₂] are relatively specific and relatively potent inhibitors of trypsin and trypsin-like enzymes.

Additional specificity and/or increased activation toward reaction with the enzyme can be introduced into the inhibitor molecule by variation of the amino acid sequence in the peptide portion of the structure. There is a generally agreement between the sequence of enzyme substrates such as a peptidyl p-nitroanilides and the sequence of an effective peptidyl phosphonate inhibitor. Inhibitors with relatively high activity generally have the sequence of a favorable peptidyl p-nitroanilide substrate for a particular enzyme. For example, a relatively potent inhibitor for chymotrypsin and chymotrypsin-like enzymes is Suc-Val-Pro-Phe^{P}(OPh)₂ which has an amino acid sequence that is analogous to Suc-Val-Pro-Phe-NA, a suitable substrate for these enzymes. With human leukocyte elastase, two relatively potent inhibitors [MeO-Suc-Ala-Ala-Pro-Val^{P}(OPh)₂ and Boc-Val-Pro-Val^{P}(OPh)₂] have an amino acid sequence similar to MeO-Suc-Ala-Ala-Pro-Val-NA and Boc-Val-Pro-Val-NA, two suitable substrates for this enzyme. For bovine thrombin, a potent phosphonate inhibitor is diphenyl Boc-D-Phe-Pro-amino(4-amidinophenyl)methanephosphonate hydrochloride, which corresponds to Boc-D-Phe-Pro-Arg-NA, which is a suitable substrate for thrombin, and D-Phe-Pro-Arg-H which is a peptide aldehyde inhibitor of thrombin and an anticoagulant (Bajusz et al., J. Med. Chem. 33:1729-1735, 1990). Since suitable substrate sequences are known in the literature for other serine proteases, it is possible to design additional phosphonate inhibitors of relatively high potency for these enzymes (Mammalian Proteases, A Glossary and Bibliography, Vol. 1 Endopeptidases, Academic Press, Barrett, A. J., and McDonald, J. K. eds, pp. 1-416, 1980; Mammalian Proteases, A Glossary and Bibliography, Vol. 2 Exopeptidases, Academic Press, Barrett, A. J., and McDonald, J. K. eds, pp. 1-357, 1986; Lottenberg et al., Meth. Enzym. 80:341-361, 1981). It is also possible to design relatively potent phosphonate inhibitors for serine proteases based on the peptide sequences found in other relatively potent reversible and irreversible inhibitors for those same serine proteases reported in the literature [Powers and Harper, in Proteinase Inhibitors, Barrett and Salvesen, eds., Elsevier, 1986, pp. 55-152; Trainor, D. A., Trends in Pharm. Sci. 8:303-307, 1987).

Diphenyl esters of α-aminoalkylphosphonate can be synthesized by previously described method (U.S. Pat. Nos. 5,543,396; 5,952,307; 5,686,419; and Oleksyszyn et al., 1979, Synthesis, 985). Di(substituted phenyl)esters of α-aminoalkylphosphonate can also be prepared by the same procedure using tris(substituted phenyl) phosphite instead of triphenyl phosphite. Perfluoroalkyl diesters can be synthesized by a method involving transesterification (Szewczyk et at., Synthesis, 409-414, 1982). Alternatively, the synthesis of diesters of α-aminoalkylphosphonic acids and their peptides can be performed by esterification of the phosphonic acid moiety as described previously (Bartlett et al., Bioorg. Chem., 14:356-377,1986). Numerous additional serine protease inhibitors that may be synthesized for use in the present invention are disclosed in U.S. Pat. Nos. 6,262,069, 5,916,888, 5,900,400, 5,157,019, 4,829,052, 5,723,316, and 5,807,829.

Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes (+) and (−) or d and 1 are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric or racemic mixture. The present invention, in certain embodiments, utilizes stereochemical or optical purity as a means for greater potency and/or decreased deleterious effects.

As used herein, the term “chiral” refers to molecules which have the property of non-superimposability of the mirror image partner, while the term “achiral” refers to molecules which are superimposable on their mirror image partner. The term “stereoisomers” refers to compounds which have identical chemical constitution, but differ with regard to the arrangement of the atoms or groups in space. In particular, “enantiomers” refer to two stereoisomers of a compound, which are non-superimposable mirror images of one another. “Diastereomers”, on the other hand, refers to stereoisomers with two or more centers of dissymmetry and whose molecules are not mirror images of one another. With respect to the nomenclature of a chiral center, the terms “S” and “R” configuration are as defined by the IUPAC 1974. Recommendations for Section E., Fundamental Stereochemistry, Pure Appl. Chem., 45:13-30 (1976).

The terms “enantiomerically enriched” and “non-racemic”, as used interchangeably herein with reference to compounds used in methods of the present invention refer to optically enriched compositions in which one enantiomer is enriched, compared to a control racemic mixture of enantiomers. Unless otherwise specified, such terms refer to compositions in which the ratio of the desired enantiomer relative to the undesied enantiomer is greater than 1:1 by weight. For instance, an enantiomerically enriched preparation has greater than 50% by weight of the preferred enantiomer relative to the udesirable enantiomer, more preferably at least 75% by weight, and even more preferably at least 80% by weight. Of course, the enrichment can be much greater than 80% by weight, providing a “substantially enantiomerically enriched”, “substantially non-racemic”, or “substantially optically pure” preparation, which refers to preparations which have at least 85% by weight of the desired enantiomer, more preferably at least 90% by weight, and even more preferably at least 95% by weight.

Separation of enantiomers can be accomplished in several ways known in the art. For example, a racemic mixture of two enantiomers can be separated by chromatography using a chiral stationary phase (see, e.g., “Chiral Liquid Chromatography”, W. J. Lough, Ed. Chapman and Hall, New York (1989)). Enantiomers can also be separated by classical resolution techniques. For example, formation of diastereometric salts and fractional crystallization can be used to separate enantiomers. For the separation of enantiomers of carboxylic acids, the diastereometric salts can be formed by addition of enantiomerically pure chiral bases such as brucine, quinine, ephedrine, strychnine, and the like. Alternatively, diastereometric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts. In addition to separation techniques such as those described above, the active enantiomer can be synthesized by stereospecific synthesis to produce only the desired optical isomer using methodology well known to those skilled in the art. Chiral synthesis can result in products of high enantiomeric purity. However, in some cases, the enantiomeric purity of the product is not sufficiently high. The skilled artisan will appreciate that the separation methods described above can be used to further enhance the enantiomeric purity obtained by chiral synthesis. The optical purity of the enantiomer can be determined by methods known in the art. For example, a sample of the enantiomer can be analyzed by high performance liquid chromatography on a chiral chromatographic column.

In one embodiment, racemic mixtures of the α-aminoalkylphosphonate derivatives may be used according to the present invention. In preferred embodiments, due to pharmacological advantages, enriched enantiomer preparations are utilized rather than racemic mixtures. In more preferred embodiments, substantially enantiomerically enriched preparations of the α-aminoalkylphosphonate derivatives are used.

C. Delivery Vehicles and Administration

The amount or concentration of protease inhibitor that can be administered is limited by efficacy at the lower end of the range and solubility of the compound at the upper end. Protease inhibitor compounds may be administered directly (e.g., topical) in at least one dose (e.g. prior to suturing) in a suitable formulation or vehicle (e.g., saline) to a site at which it is desired to prevent or reduce adhesion formation (e.g., abdominal, thoracic, ophthalmic, cardiac, or gynecologic tissue). In one embodiment, at least one protease inhibitory compound is administered as a single dose, such as prior to suturing post-surgery, using a delivery vehicle or system suitable for sustained release of the compound at a requisite concentration for a period of time sufficient for re-epithelialization. Preferably the delivery vehicle or system itself is relatively inert, in that it is relatively non-immunogenic and relatively non-inflammatory, while permitting sustained release of the protease inhibitor at effective treatment levels over the desired time period. A variety of delivery systems and formulations for sustained release are well known in the art and contemplated for use in the present invention. Specific examples of such delivery vehicles and formulations include, but are not limited to, liposomes and other lipid-based systems, microspheres and microcapsules, biodegradable mechanical barriers, bioadhesives, viscous instillates, and polymeric materials, such as polymethacrylate and polymethacrylamide hydrogels, polyanhydrides, poly-orthoesters, polyethylene oxide and polypropylene oxide block copolymers, carbohydrate polymers, and the like, such as described in U.S. Pat. No. 4,937,254.

One embodiment utilizes microsphere or microcapsule delivery vehicles prepared from a biodegradable, sterilizable and non-toxic polymer, such as, for example, a polymer used in adsorbable suture material. Polymers for use in the present invention include, without limitation, poly(α-hydroxy acid), polyhydroxybutyric acid, polycaprolactone, polyorthoester, polyanhydride, PACA, and polycyanoacrylate. Preferably, microspheres or microcapsules for use with the present invention are polymer microspheres or microcapsules derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The polymer microspheres or microcapsules may be derived from any of various polymeric starting materials which have a variety of molecular weights and, in the case of the copolymers such as PLG, a variety of lactide:glycolide ratios. Encapsulation of a variety of prophylactic and therapeutic compounds is known in the art and has been reported, for example using poly(D,L-lactide-co-glycolide).

In another embodiment, films or barriers, particularly those which are prepared from a biodegradable, sterilizable and non-toxic polymer, such as, for example, a polymer used in adsorbable suture material, are utilized. Polymers for use in the present invention include, without limitation, poly(α-hydroxy acid), polyhydroxybutyric acid, polycaprolactone, polyorthoester, polyanhydride, PACA, and polycyanoacrylate. Preferably, films or barriers for use with the present invention are polymers derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. Such films or barriers are well known in the art. While adhesion prevention barriers, such as Seprafilm and Interceed, are being incorporated into clinical practice as a “stand-alone” methodologies, their effectiveness is not universal and the further use of a protease inhibitor of the present invention should provide the requisite level of effectiveness in patients.

In another embodiment, an aqueous composition which can be applied at room temperature as a liquid, but which forms a semi solid gel when warmed to body temperature is utilized as a vehicle for drug delivery. Such a system combines ease of application with greater retention at the site requiring treatment than would be the case if the aqueous composition were not converted to a gel as it is warmed to mammalian body temperature. For example, as described in U.S. Pat. No. 4,188,373, polyols may used in aqueous compositions to provide thermally gelling aqueous systems. Adjusting the concentration of the polymer, provides the desired sol-gel transition, that is, the lower the concentration of polymer, the higher the sol-gel transition temperature. Alternatively, as described in U.S. Pat. Nos. 4,474,751; 752; 753; and 4,478,822, drug delivery systems which utilize thermosetting polyoxyalkylene gels may be used, as well as other aqueous gel compositions, such as for example, as described in U.S. Pat. Nos. 4,883,660; 4,861,760; 4,810,503; 4,767,619; and 4,511,563.

Another embodiment for the delivery of at least one protease inhibitor of the present invention, utilizes liposomes or lipid-based carriers or delivery vehicles. The encapsulation of an active agent in multilamellar vesicles (or liposomes) is a technique used to assist in target drug delivery and prolong drug residence. In a typical procedure, a liposome-forming powdered lipid mixture is added to the desired quantity of active agent in aqueous solution, for example, phosphate buffered saline, to form a suspension. A lipid mixture suitable for formation of liposomes may be prepared from L-alpha-distearoyl phosphatidylcholine and cholesterol dissolved in chloroform, to which alpha-tocopherol is added; other compositions and methods for formation of liposomes would, however, also be useful for this purpose. The intraperitoneal administration of liposomes containing ibuprofen or tolmetin is described in Rodgers, K. et al., “Inhibition of Postsurgical Adhesions by Liposomes Containing Nonsteroidal Anti-inflammatory Drugs,” Int. J. Fertil., Vol. 35, p. 40 (1990).

In yet another embodiment, the delivery of at least one protease inhibitor of the present invention utilizes high-molecular-weight carriers used in admixture with the active agents. Examples of such carriers include, but are not limited to, the following: dextrans and cyclodextrans; hydrogels; cross-linked viscous materials, including viscoelastics and cross-linked viscoelastics; carboxymethylcellulose; and hyaluronic acid.

Pursuant to the method of the present invention, at least one protease inhibitor is maintained in an effective concentration at the site of potential adhesion formation for a period of time sufficient to permit substantial re-epithelialization. The protease inhibitor compound is typically administered over the perioperative interval, which for purposes of the present invention may include a period of time prior to surgery, through surgery itself, and also post-completion of surgery. The term of administration of administration may vary, but typically should be effected from the time of surgery for at least 24 to 48 hours after completion of surgical procedures. In general, the protease inhibitory compound may be administered, with or without sustained release vehicles or formulations, from the time of surgery for a period ranging between about 24 hours and about 14 days (a period that generally will include complete healing). Preferably the compound may be administered as above from the time of surgery for a period ranging between about 24 hours and 7 days, and more preferably ranging between about 24 hours and 3 days. For example, using one protease inhibitor of the present invention, the chymase inhibitor Suc-Val-Pro-Phe^{P}(OPh)₂, chymase activity was significantly suppressed in vascular tissues for 4 weeks, when 10 μM administration was used only during the operation. For the experiments outlined below, it is thought that chymase activity in the injured uterus might be inhibited even 14 days after the operation. A range of therapeutic delivery doses are contemplated for the present invention. For example, for the chymase inhibitor, Suc-Val-Pro-Phe^{P}(OPh)₂, an effective delivery dosage may be from 0.01 μM to 100 μM. Preferably the delivery dosage is from 0.1 μM to 10 μM, and more preferably the delivery dosage is 0.5 μM to 5 μM. Effective dosages, regimens, and routes of administration for other protease inhibitors may be readily determined by one of skill in the art using the teachings provided herein.

The following examples are included to more fully illustrate the present invention. Additionally, these examples provide some embodiments of the invention and are not meant to limit the scope thereof.

### EXAMPLES

### Example 1

### Preparation of α-Aminoalkylphosphonate Derivatives

Preparation of peptidyl derivatives of aryl diesters of α-aminoalkylphosphonic acids has been documented and may be performed by one of skill in the art (see, for example U.S. Pat. No. 5,686,419). To elucidate the relationship between chymase activity and adhesion formation, the effect of a representative serine protease inhibitor, the chymase inhibitor, Suc-Val-Pro-Phe^{P}(OPh)₂, was tested. This compound may be synthesized using published methodologies (U.S. Pat. No. 5,686,419; Oleksyszyn and Powers, Biochem., 30:485-493, 1991). More specifically, the reaction of Cbz-Val-OH (0.25 g, 1 mmol), DCC (0.2 g, 1 mmol) and the product of hydrogenolysis of Cbz-Pro-Phe^{P}(OPh)₂ (0.584 g, 1 mmol) gives an oil which is dissolved in 30 ml of ethyl acetate. To this solution, 0.1 g (1 mmol) of succinic anhydride and 0.1 g of 5% Pd/C is added and mixture is stirred under atmosphere of hydrogen until thin layer chromatography (TLC) shows only one new spot. Catalyst is removed by filtration and the organic layer is washed several times with water. After drying, the organic solvent is removed to give, for example, 0.45 g (65%) of product as a hydroscopic solid: mp. 50°-53° C.; one spot on TLC, R_{f}=0.4; ³¹P NMR 19.75, 19.23 ppm, ratio 1:1. Anal. Calcd. for C₃₄H₄₀O₃N₃P.2H₂O: C, 59.56; H, 6.42. Found: C, 59.59; H, 6.42.

### Example 2

### Preparation of Enriched Enantiomers of Chymase Inhibitor Suc-Val-Pro-Phe^{P}(OPh)₂

As used below, the following abbreviations apply: Z is Benzyloxycarbonyl, Boc is tert-butyloxycarbonyl, WSCD is Carboduimide, and HOBt is 1-Hydroxybenzotriazol. Z-Phe^{P}(OPh)₂ was synthesized according to the procedure of Oleksyszyn and Powers (Methods Enzymol. 244:423-441, 1994) and benzyloxycarbonyl was removed by hydrogen bromide/acetic acid solution. The sample was coupled with Boc-Pro by WSCD-HOBt reaction and then the racemic mixture was obtained. The racemic mixture then was separated by re-precipitation. Inactive enantiomer was firstly crystallized from the solution and removed. After de-blocking of Boc with HCl from the active enantiomer in the solution, the sample was coupled with Boc-Val by WSCD-HOBt reaction and deblocked Boc again. To this product, succinic anhydride and triethyamine were added and Suc-Val-Pro-Phe^{P}(OPh)₂ was obtained. The product was finally enriched by reverse phase HPLC. More specifically, detailed synthesis of the enantiomerically enriched preparation was performed as follows:

Z-DL-Phe^{P}(OPh)₂

Phenylacetaldehyde (28.3 mL, 0.242 mol) was dissolved in 45 mL of acetic acid. Carbamic acid benzyl ester (24.4 g, 0.161 mol) and triphenyl phosphite (50.0 g, 0.161 mol) were added to this solution and stirred for 1.5 h at 85 ° C. After the organic solvent were evaporated and the residual solution was cooled to the room temperature, 400 mL of methanol was added to this solution and it was allowed to crystallize by friction at −20 ° C. The product was collected by filtration, washed with cold methanol and dried in vacuo to yield 32.9 g (42%) of mixture of Z-D-Phe^{P}(OPh)₂ and Z-L-Phe^{P}(OPh)₂.

DL-Phe^{P}(OPh)₂.HBr

The mixture of Z-D-Phe^{P}(OPh)₂ and Z-L-Phe^{P}(OPh)₂ (14.3 g, 29.3 mmol) was dissolved in 30 mL of 25% hydrogen bromide/acetic acid solution and stirred for 1 h at room temperature. After addition of ether, the separated solid sample was collected by filtration. This sample was washed with ether and dried in vacuo to yield 12.0 g (94%) of the mixture of D and L-Phe^{P}(OPh)₂.HBr.

Boc-Pro-DL-Phe^{P}(OPh)₂

The mixture of D and L-Phe^{P}(OPh)₂.HBr (11.5 g, 26.5 mmol), Boc-Pro (5.53 g, 25.7 mmol) and HOBt (3.58 g, 26.5 mmol) were dissolved in 70 mL of DMF and WSCD (4.70 mL, 26.5 mmol) was added drop-wise into this solution under cooled with ice. After being stirred for 3.5 h at room temperature, the solution was concentrated in vacuo and ethyl acetate was added. The resulting solution was successively washed with acidic and alkaline solution, and dried over MgSO₄, filtered and concentrated in vacuo. The inactive enantiomer was crystallized from acetone-ether and removed. The residual solution was concentrated in vacuo and purified by midium-pressure silica-gel chromatography with toluen-ethyl acetate (5:1) as eluent to yield 5.10 g of the active enantiomer (A).

Boc-Val-Pro-Phe^{P}(OPh)₂-A

Boc-Pro-Phe^{P}(OPh)₂-A (4.98 g, 9.05 mmol) was dissolved with 37 mL of cold HCl/dioxane (4.9 N) and stirred for 1 h at room temperature. The solution was evaporated and dried in vacuo. The residue was dissolved with 40 mL of DMF, and Boc-Val (2.06 g, 9.50 mmol) and HOBt (1.35 g, 9.96 mmol) were added to this solution. WSCD (1.77 mL, 9.96 mmol) was added dropwise into this solution under cooled with ice and stirred over night at room temperature. Ethyl acetate was added to this reacting solution and successively washed with acidic and alkaline solution. After being dried over MgSO₄ and filtered, solution was concentrated in vacuo to yield 6.26 g (94%) of product, as a colorless oil.

Suc-Val-Pro-Phe^{P}(OPh)₂-A

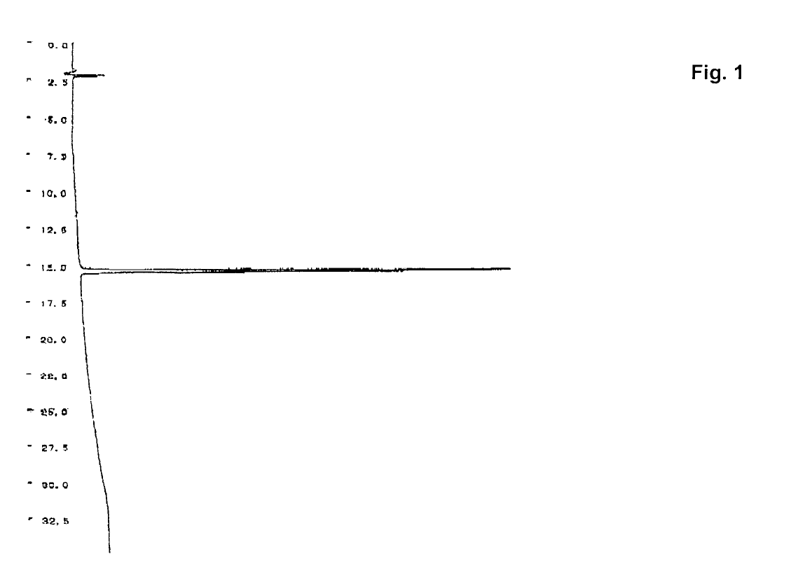

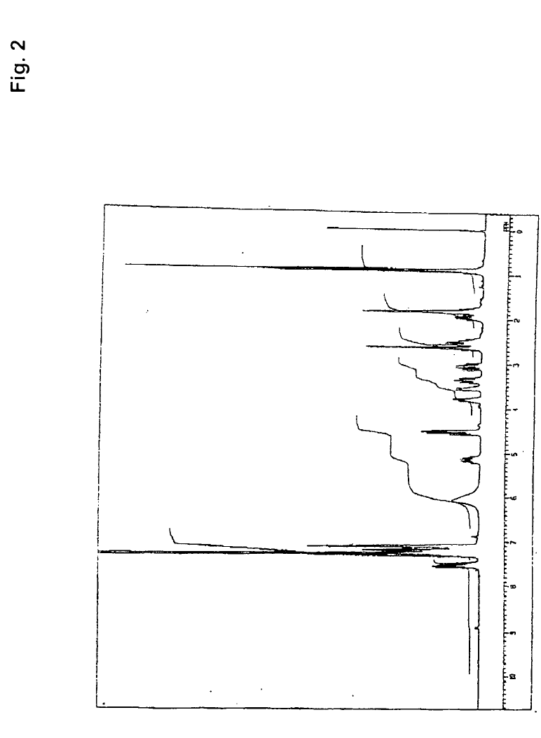

Boc-Val-Pro-Phe^{P}(OPh)₂-A (5.86 g, 8.47 mmol) was dissolved with 35 mL of cold HCl/dioxane (4.9 N) and stirred for 1 h at room temperature. The solution was evaporated and dried in vacuo. The residue was dissolved with 35 mL of DMF, and succinic anhydride (1.02 g, 10.2 mmol) was added. Triethylamine (2.36 mL, 16.9 mmol) was added drop-wise into this solution and stirred for 2 h at room temperature. Under cooled with ice, pH of solution was adjusted to pH 1.0 by HCl (1 mol) and the sample was extracted with ethyl acetate. The extract was washed with saturated saline solution, dried over MgSO₄, filtered and concentrated ini vacuo to yield the product, as a yellow oil. The resulting oil was enriched by the reverse-phase HPLC (Column: YMC ODS SH-363-5, 30×250 mm, Mobile phase: 0.1% TFA gradient 40-70% MeCN) and freeze-dried to yield 2.30 g (42%) of product as a white powder. FIGS. 1 and 2 illustrate purification of the enantiomer Suc-Val-Pro-Phe^{P}(OPh)₂-A.

Results of Analysis

Sample: Suc-Val -Pro-Phe^{P}(OPh)₂-A

LotNo.: 520217

Volume: 2.0 g×1

Appearance: White powder

Purity (by HPLC): Main peak 98.8% (Attachment 1)

HPLC conditions

Column: YMC Pack, ODS-A 4.6 mm I.D.×150 mm

Mobile phase: 0.1% TFA, gradient 30-80% MeCN (25 min)

Flow rate: 1.0 mL/min

Detector: An ultraviolet absorption photometer (wavelength: 220 nm)

Amino-acid analysis:

| | mol ratio | recovery |
|---|---|---|
| Val (1) | 1.00 | 90.5% |
| Pro (1) | 1.03 | |
| Phe^{p} (OH)₂ | 0.98 | |

Hydrolysis conditions: 6 mol HCl with phenol, 110°C., 22 hours

Elemental analysis: Calcd for C₃₄H₄₀N₃O₈S:C, 62.86; H, 6.21; N, 6.47%; Calcd for C₃₄H₄₀N₃O₈S.8H₂O.0.5TFA:C, 58.30; H, 5.88; N, 5.83%; Found:C, 58.34; H, 5.94; N, 5.51%; Mass spectrometry: 650.3 (Calcd [M+H]_{exact}=650.253 (by ESI−MS); ¹H-NMR spectrometry: Attachment 2.

### Example 3

### Surgical Technique for Treatment Evaluation

Mature female Syrian hamsters (SLC, Shizuoka, Japan), 6 weeks of age, weighing 85 to 90 g were maintained in an environmentally controlled room with a 12-hour light, 12-hour dark cycle. Hamsters were anesthetized with intraperitoneal sodium pentobarbital (50 mg/kg). Surgical technique was performed using a modified procedure of Nagler et al. (Am. J. Obstet. Gynecol. 180:558-63, 1999). An abdominal midline incision was made, and the right uterus was grasped and denuded of serosa over half the length of the uterine body until punctate hemorrhage occurred, using a swab. In the chymase-treated group, 1 ml of 10 μM Suc-Val-Pro-Phe^{P}(OPh)₂ in saline was injected into the abdomen. Then, the abdomen was closed in two layers with silk sutures. In the placebo group, the same 1 ml amount of saline was administered as in the chymase inhibitor group.

Three days after the surgery, the animals (placebo group, n=13; chymase group, n=13) were anesthetized with sodium pentobarbital (50 mgikg, i.p.), and then the uterus was removed for the measurement of chymase activity. Two weeks after the surgery, the animals (placebo group, n=15; chymase group, n=16) were anesthetized with sodium pentobarbital (50 mg/kg, i.p.), and then adhesions were assessed.

### Example 4

### Histological Study

Portions of the adhesion lesions were fixed in Carnoy's solution, embedded in paraffin, and cut into 3-μm thick sections. The specimens were stained with hematoxylin and eosin. For determination of mast cells, the specimens were stained with toluidine blue. To determine the localization of chymase, immunohistochemistry was performed by labeling the chymase in situ with a streptavidin-biotin peroxidase kit (Dako LSAB kit, DAKO Corporation, Carpinteria, Calif.), using a polyclonal antibody (1:100 dilution) for hamster chymase as the primary antibody (Jin et al., Jpn. J. Pharmacol. 86:203-14, 2001).

### Example 5

### Tissue Preparation

A tissue extract for measurement of chymase activity was prepared as described previously (Takai et al., Life Sci. 58:591-597, 1996). The uterus was minced and homogenized in 10 volumes (w/v) of 20 mM Na-phosphate buffer (pH 7.4). The homogenate was centrifuged at 10,000 rpm for 30 minutes. The supernatant was discarded. These steps were repeated twice. The pellets were re-suspended and homogenized in 5 volumes (w/v) of 10 mM Na-phosphate buffer (pH 7.4) containing 2 M KCl and 0.1% Nonidet P-40. The homogenate was stored overnight at 4° C., and then centrifuged at 10,000 rpm for 30 minutes. The supernatant was used as the tissue extract for the measurement of chymase activity.

### Example 6

### Measurement of Chymase Activity

Chymase activity was measured as described previously (Jin et al., J. Hypertens. 16:657-664, 1998). Aliquots (20 μl) of tissue extract were incubated for 10 minutes at 37° C. with 4 mM angiotensin (Ang) I in 150 mM borax-borate buffer, pH 8.5 (final incubation volume of 0.1 ml) containing 5 mM ethylenediaminetetraacetic acid, 8 mM dipyridyl and 0.77 mM diisopropyl phosphorofluoridate. Reactions were terminated by addition of 0.15 ml of 15% trichloroacetic acid, and then centrifugation was conducted at 10,000 rpm for 10 minutes. For fluorometric quantification of His-Leu as an Ang II metabolite, 10% o-phthaldialdehyde (dissolved in neat methanol) was added to the supernatant under alkaline conditions, and then 6 N HCl was added to stabilize the fluorescence for the measurement at 340 nm excitation and 455 nm emission. A blank was carried out with the addition of 0.5 mM chymostatin (dimethylformamide solution). Protein concentration was measured with the bicinchoninic acid protein assay reagent (Pierce Chemical, Rockford, Ill.) using bovine serum albumin as a standard.

### Example 7

### Scoring of Adhesions and Statistical Analysis

The adhesions were graded blindly according to a modified classification of Hulka et al. (Fertil Steril. 30:661-665, 1978): Score 0, no adhesions; Score 1, mild adhesions; Score 2, localized moderate adhesions; Score 3, moderate and wide adhesions; Score 4, severe adhesions, and impossible to separate. Chymase activities were evaluated in a parametric test using Fisher's Protected Least Significant Difference. Adhesion scores were evaluated in a nonparametric test, and statistically analyzed by the Mann-Whitney's U test. Values are given as means ±standard error (S.E.). Differences were considered statistically significant at P<0.05.

### Example 8

### Treatment Results

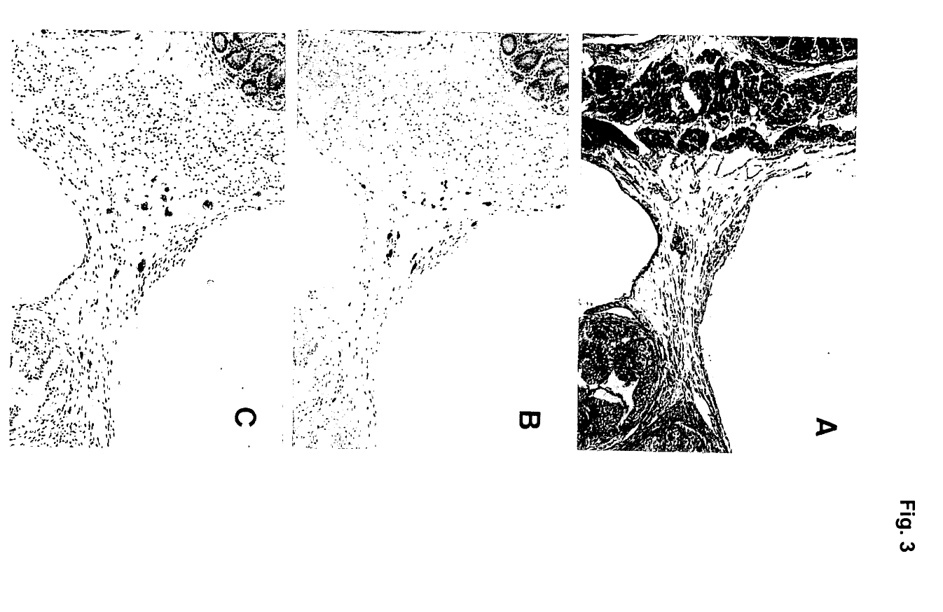

A typical adhesion lesion between the oviduct and the small intestine stained with hematoxylin-eosin is shown in FIG. 3A. In this lesion, positive cells stained with toluidine blue, which stains mast cells, were observed and these were also identified as positive cells by immunostaining with the hamster chymase antibody (FIGS. 3B and 3C).

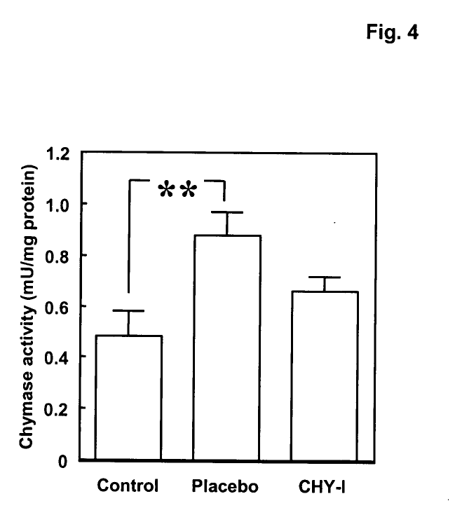

As shown in FIG. 4, the uterus chymase activity in the placebo-treated hamsters three days after the operation was significantly increased in comparison with that of control hamsters (control hamsters, 0.48±0.10 mU/mg protein; placebo-treated hamsters, 0.88±0.09 mU/mg protein, P=0.002). However, the uterus chymase activity in the hamsters treated with the chymase inhibitor was 0.66±0.06 mU/mg protein, and this value was decreased in comparison with the value in the placebo-treated hamsters (P=0.06).

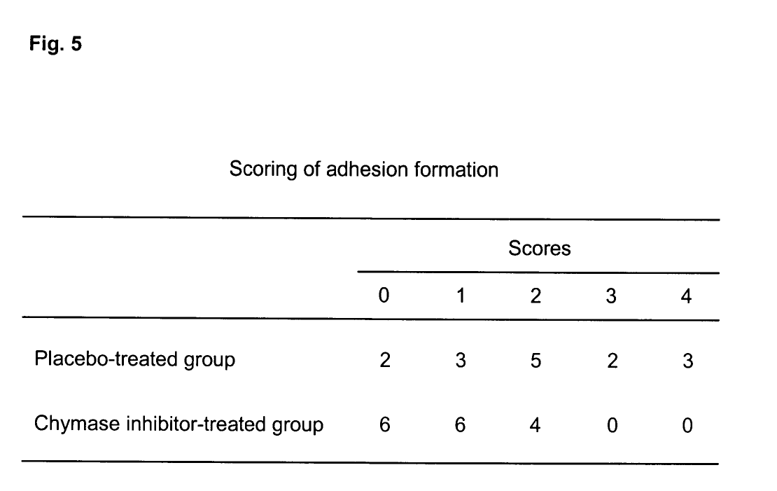

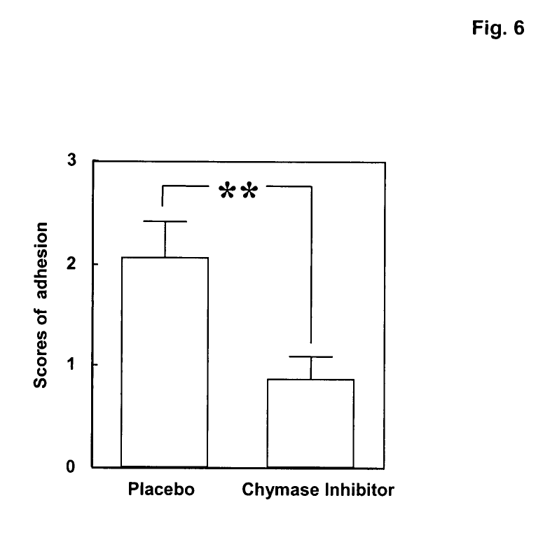

In the placebo-treated hamsters, scores of adhesion formation between 0 and 4 were observed, while in the hamsters treated with the chymase inhibitor, values no higher than 3 were found (FIG. 5). The scores of adhesion formation in hamsters treated with placebo and the chymase inhibitor were 2.07±0.34 and 0.88±0.20, respectively, and this difference is significant (FIG. 6). Subsequent experiments in this animal model using enantiomerically enriched preparations of Suc-Val-Pro-Phe^{P}(OPh)₂ demonstrated that the enriched enantiomer Suc-Val-Pro-Phe^{P}(OPh)₂-A, but not enantiomer Suc-Val-Pro-Phe^{P}(OPh)₂-I, resulted in a significant preventative effect, reducing adhesion formation.

Therefore, these examples establish that chymase plays an important role in the development of adhesion formation, and a protease inhibitor that exhibits inhibitory effects on chymase, for example Suc-Val-Pro-Phe^{P}(OPh)₂, may be a useful drug for prevention or inhibition of adhesion formation, such as for example peritoneal adhesions. In addition, other compounds which inhibit protease (e.g., chymase) activity and inhibit or prevent adhesion formation may be identified using the teachings provided herein.

All references including publications, patent applications and patents cited herein are hereby incorporated by reference to the same extent as if each reference was individually and specifically indicated to be incorporated by reference and were set forth in its entirety herein.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for preventing or reducing adhesion formation between tissue surfaces in a vertebrate subject, comprising administering to the subject an effective amount of at least one protease inhibitor to a site on a tissue surface for a period of time sufficient to prevent or reduce adhesion formation.

2. A method according to claim 1, wherein said protease inhibitor is an inhibitor of a serine protease.

3. A method according to claim 2, wherein said inhibitor of a serine protease is an inhibitor of a chymotrypsin-like serine protease.

4. A method according to claim 3, wherein said inhibitor of a chymotrypsin-like serine protease is an inhibitor of a chymase.

5. A method according to claim 4, wherein said inhibitor of a chymase is a peptidyl derivative of aryl diesters of α-aminoalkylphosphonic acids.

6. A method according to claim 4, wherein said inhibitor of a chymase is Suc-Val-Pro-Phe^{P}(OPh)₂.

7. A method according to claim 4, wherein said inhibitor of a chymase is an enantiomerically enriched preparation of Suc-Val-Pro-Phe^{P}(OPh)₂.

8. A method according to claim 7, wherein Suc-Val-Pro-Phe^{P}(OPh)₂-A comprises greater than 50% by weight of the total Suc-Val-Pro-Phe^{P}(OPh)₂ in said enantiomerically enriched preparation.

9. A method according to claim 7, wherein Suc-Val-Pro-Phe^{P}(OPh)₂-A comprises greater than 80% by weight of the total Suc-Val-Pro-Phe^{P}(OPh)₂ in said enantiomerically enriched preparation.

10. A method according to claim 7, wherein Suc-Val-Pro-Phe^{P}(OPh)₂-A comprises greater than 95% by weight of the total Suc-Val-Pro-Phe^{P}(OPh)₂ in said enantiomerically enriched preparation.

11. A method according to claim 1, wherein said protease inhibitor is administered to said subject before, during or after a surgical procedure.

12. A method according to claim 11, wherein said surgical procedure is an abdominal surgical procedure.

13. A method according to claim 11, wherein said surgical procedure is a thoracic surgical procedure.

14. A method according to claim 11, wherein said surgical procedure is an ophthalmic surgical procedure.

15. A method according to claim 11, wherein said surgical procedure is a cardiac or gynecologic surgical procedure.

16. A method for preventing or reducing postoperative adhesion formation in the peritoneum of a warm-blooded mammal, comprising administering to said mammal an effective amount of at least one serine protease inhibitor to a site on an organ surface for a period of time sufficient to prevent or reduce adhesion formation.

17. A method according to claim 16, wherein said serine protease inhibitor is an inhibitor of a chymotrypsin-like serine protease.

18. A method according to claim 17, wherein said inhibitor of a chymotrypsin-like serine protease is an inhibitor of a chymase.

19. A method according to claim 18, wherein said inhibitor of a chymase is a peptidyl derivative of aryl diesters of α-aminoalkylphosphonic acids.

20. A method according to claim 18, wherein said inhibitor of a chymase is Suc-Val-Pro-Phep^{P}(OPh)₂.

21. A method according to claim 18, wherein said inhibitor of a chymase is an enantiomerically enriched preparation of Suc-Val-Pro-Phe^{P}(OPh)₂.

22. A method according to claim 21, wherein Suc-Val-Pro-Phe^{P}(OPh)₂-A comprises greater than 50% by weight of the total Suc-Val-Pro-Phe^{P}(OPh)₂ in said enantiomerically enriched preparation.

23. A method according to claim 21, wherein Suc-Val-Pro-Phe^{P}(OPh)₂-A comprises greater than 80% by weight of the total Suc-Val-Pro-Phe^{P}(OPh)₂ in said enantiomerically enriched preparation.

24. A method according to claim 21, wherein Suc-Val-Pro-Phe^{P}(OPh)₂-A comprises greater than 95% by weight of the total Suc-Val-Pro-Phe^{P}(OPh)2 in said enantiomerically enriched preparation.

25. A method according to claims 1, wherein said protease inhibitor is administered in conjunction with a delivery vehicle which maintains an effective local concentration of said protease inhibitor at said site, and wherein said delivery vehicle comprises microcapsules or microspheres.

26. A method according to claim 25, wherein said microcapsules or microspheres comprise a biodegradable polymer selected from the group consisting of poly(α-hydroxy acids), polyhydroxybutyric acids, polycaprolactones, polyorthoesters, polyanhydrides, PACA, polycyanoacrylates, poly(D,L-lactide-co-glycolide) and mixtures thereof.

27. A method according to claims 1, wherein said protease inhibitor is administered in conjunction with a delivery vehicle which maintains an effective local concentration of said protease inhibitor at said site, and wherein said delivery vehicle comprises a film.

28. A method according to claim 27, wherein said film comprise a biodegradable polymer selected from the group consisting of poly(α-hydroxy acids), polyhydroxybutyric acids, polycaprolactones, polyorthoesters, polyanhydrides, PACA, polycyanoacrylates, poly(D,L-lactide-co-glycolide) and mixtures thereof.

29. A method according to claims 1, wherein said protease inhibitor is administered in conjunction with a delivery vehicle which maintains an effective local concentration of said protease inhibitor at said site, and wherein said delivery vehicle comprises liposomes.

30. A method according to claims 1, wherein said protease inhibitor is administered in conjunction with a delivery vehicle which maintains an effective local concentration of said protease inhibitor at said site, and wherein said delivery vehicle comprises a high-molecular weight carrier selected from the group consisting of hyaluronic acid, hydrogels, carboxymethlcellulose, dextrans, cyclodextrans, and mixtures thereof.

31. A method according to claim 1, wherein said vertebrate subject is a human.

32. A method according to claim 16, wherein said warm-blood mammal is a human.

33. A pharmaceutical composition for the prevention of adhesion formation, comprising the protease inhibitor of any one of claims 1-24 and a pharmaceutically acceptable diluent or excipient.

34. A pharmaceutical composition according to claim 33, further comprising a delivery vehicle which maintains an effective local concentration of said protease inhibitor at a site on an tissue surface for a period of time sufficient to prevent or reduce adhesion formation.
